(19) United States
(12) Patent Application Publication
(10) Pub. No.: US 2007/0161670 A1
(43) Pub. Date: Jul. 12, 2007

(54) PROCESS FOR THE PREPARATION OF SUBSTITUTED HETEROCYCLES

(75) Inventors: Andrew J. Staab, Middletown, CT (US); Ny Sin, East Hampton, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/619,656

(22) Filed: Jan. 4, 2007

Related U.S. Application Data
(60) Provisional application No. 60/757,257, filed on Jan. 9, 2006.

(51) Int. Cl. A61K 31/4745; A61K 31/4704; C07D 471/02; C07D 217/22

(52) U.S. Cl. 514/300; 514/309; 546/122; 546/143

(57) ABSTRACT

The present disclosure generally relates to a process for the preparation of hydroxy-substituted heterocycles such as isoquinolines, naphthyridines, pyridopyridazines, and pyridopyrimidines.

### CROSS-REFERENCE TO RELATED APPLICATION

This present application is related to U.S. provisional application Ser. No. 60/757,257 filed Jan. 9, 2006, the contents of which are incorporated by reference herein in their entirety.

The present disclosure generally relates to a process for the preparation of hydroxy-substituted heterocycles such as isoquinolines, naphthyridines, pyridopyridazines, and pyridopyrimidines.

Compound (5) is an intermediate used in the preparation of phenylglycinamide derivatives (for example, Compound (6)) which are useful in the treatment of thrombotic disease.

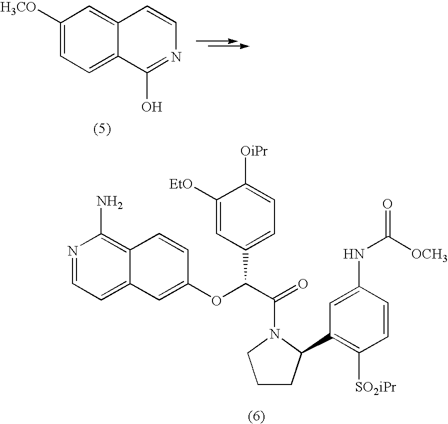

The previously disclosed method for the formation of compound (5) (see WO 2005/054430) is impractical for large-scale production as it uses sodium azide and requires temperatures of over 200° C. Thus, there is a continuing need for methods of preparing hydroxy-substituted heterocycles such as compound (5) that are amenable to large-scale production.

In a first aspect the present disclosure provides a process for preparing a compound of formula (4)

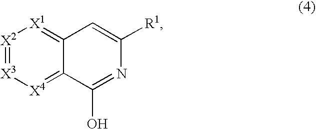

wherein

X¹, X², X³, and X⁴ are independently N or CR²; provided that if X¹ and X² are N then X³ is CR²; and provided that if X³ and X⁴ are N then X² is CR²;

R¹ is selected from hydrogen and —NR^{a}R^{b};

each R² is independently selected from alkenyl, alkoxy, alkoxyalkyl, alkyl, alkynyl, aryl, arylalkyl, halo, haloalkoxy, haloalkyl, heterocyclyl, heterocyclylalkyl, —NR^{a}R^{b}, and (NR^{a}R^{b})alkyl; and

R^{a} and R^{b} are independently selected from hydrogen, alkenyl, alkyl, alkynyl, aryl, and arylalkyl;

the process comprising:
(a) reacting a compound of formula (3)

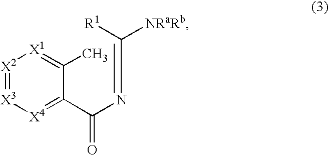

with a base to form a first reaction mixture;
(b) adjusting the pH of the first reaction mixture to about 7 to form a second reaction mixture; and
(c) optionally heating the second reaction mixture.

In a first embodiment of the first aspect step (a) is conducted in an organic solvent. In a second embodiment of the first aspect the organic solvent is an ether. In a third embodiment of the first aspect the organic solvent is tetrahydrofuran.

In a fourth embodiment of the first aspect the base is selected from potassium tert-butoxide, lithium tert-butoxide, sodium tert-butoxide, lithium hexamethyldisilazide, lithium diisopropylamide, and sodium hexamethyldisilazide. In a fifth embodiment of the first aspect the base is potassium tert-butoxide. In a sixth embodiment of the first aspect the base is lithium diisopropylamide.

In a seventh embodiment of the first aspect step (a) is conducted at a temperature of about 0° C. to about 70° C. In an eighth embodiment of the first aspect step (a) is conducted for about 15 minutes to about 2 hours.

In a ninth embodiment of the first aspect the pH of the first reaction mixture is adjusted to about pH 7 with hydrochloric acid. In a tenth embodiment of the first aspect the pH of the first reaction mixture is adjusted to about pH 7 with ammonium chloride.

In an eleventh embodiment of the first aspect step (b) is conducted at a temperature of about 20° C. to about 40° C.

In a twelfth embodiment of the first aspect step (c) is conducted at a temperature of about 50° C. to about 70° C.

In a thirteenth embodiment of the first aspect step (c) is conducted for about 15 minutes to about 2 hours.

In a second aspect the present disclosure provides a process for preparing a compound of formula (4a)

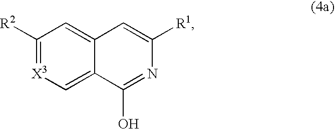

wherein

R¹ is hydrogen or —N(CH₃)₂;

R² is —OCH₃ or —N(CH₃)₂; and

X³ is CH or N;

the process comprising:
(a) reacting a compound of formula (3a)

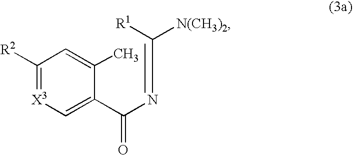

with a base to form a first reaction mixture;
(b) adjusting the pH of the first reaction mixture to about 7 to form a second reaction mixture; and
(c) optionally heating the second reaction mixture.

In a first embodiment of the second aspect

R¹ is hydrogen;

R² is —OCH₃; and

X³ is CH.

As used in the present specification, the following terms have the meanings indicated:

As used herein, the singular forms “a”, “an”, and “the” include plural reference unless the context clearly dictates otherwise.

The term “alkenyl,” as used herein, refers to a straight or branched chain group of two to six carbon atoms containing at least one carbon-carbon double bond.

The term “alkoxy,” as used herein, refers to an alkyl group attached to the parent molecular moiety through an oxygen atom.

The term “alkoxyalkyl,” as used herein, refers to an alkyl group substituted with one, two, or three alkoxy groups.

The term “alkyl,” as used herein, refers to a group derived from a straight or branched chain saturated hydrocarbon containing from one to ten carbon atoms.

The term “alkynyl,” as used herein, refers to a straight or branched chain hydrocarbon of two to six carbon atoms containing at least one carbon-carbon triple bond.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present invention can be attached to the parent molecular moiety through any substitutable carbon atom in the group. Representative examples of aryl groups include, but are not limited to, indanyl, indenyl, naphthyl, phenyl, and tetrahydronaphthyl.

The term “arylalkyl,” as used herein, refers to an alkyl group substituted with one, two, or three aryl groups.

The term “base,” as used herein, refers to a reagent capable of accepting protons during the course of a reaction. Examples of bases useful in the processes of the present disclosure include, but are not limited to, lithium hexamethyldisilazide, sodium hexamethyldisilazide, potassium hexamethyldisilazide, lithium diisopropylamine, potassium tert-butoxide, sodium tert-butoxide, and lithium tert-butoxide.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heterocyclyl,” as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a phenyl group, a monocyclic cycloalkenyl group, a monocyclic cycloalkyl group, or another monocyclic heterocyclyl group; and tricyclic groups in which a bicyclic system is fused to a phenyl group, a monocyclic cycloalkenyl group, a monocyclic cycloalkyl group, or another monocyclic heterocyclyl group. The heterocyclyl groups of the present invention can be attached to the parent molecular moiety through a carbon atom or a nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylalkyl,” as used herein, refers to an alkyl group substituted with one, two, or three heterocyclyl groups.

The term “—NR^{a}R^{b},” as used herein, refers to two groups, R^{a} and R^{b}, which are attached to the parent molecular moiety through a nitrogen atom. R^{a} and R^{b} are independently selected from hydrogen, alkenyl, alkyl, alkynyl, aryl, and arylalkyl.

The term “—R^{a}R^{b})alkyl,” as used herein, refers to an alkyl group substituted with one, two, or three NR^{a}R^{b} groups.

The term “organic solvent,” as used herein, refers to an organic substance that is a liquid at between about 20° C. and about 35° C. and does not interact with starting materials, reagents, intermediates, or products in a manner which adversely affects the yield of the desired product.

All of the processes of the invention can be conducted as continuous processes. The term “continuous process,” as used herein, refers to the conduction of steps without isolation of the intermediates.

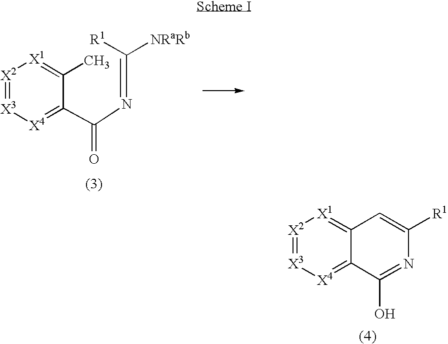

Scheme I shows the methodology of the present disclosure. The compound of formula (3), which can be prepared by methods as described herein or by methods known to those of ordinary skill in the art, can be converted to compounds of formula (4) by treating with base followed by neutralization and optional heating. The particular base, quenching agent, and temperature used will depend on the identity of R¹. Representative bases include potassium tert-butoxide, sodium tert-butoxide, lithium tert-butoxide, lithium diisopropylamide, lithium hexamethyldisilazide, sodium hexamethyldisilazide, and potassium hexamethyldisilazide. Examples of quenching agents include hydrochloric acid, ammonium chloride, and sulfuric acid. Reaction temperatures range from 0° C. to about 80° C. and reaction times range from about 1 to about 18 hours.

The present disclosure will now be described in connection with certain embodiments which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

### EXAMPLES

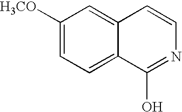

### Example 1

### Preparation of 6-methoxyisoquinolin-1-ol

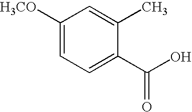

### Example 1A

### 4-methoxy-2-methylbenzoic acid

A flask equipped with a mechanical stirrer, reflux condenser, and addition funnel was charged with magnesium (61.4 g) and THF (1 L) and put under a nitrogen atmosphere. The magnesium was treated with approximately 5-10% 4-bromo-3-methylanisole and the reaction flask was warmed to 40° C. until the reaction was well initiated. The remaining 4-bromo-3-methylanisole (90-95%, 500 mg total amount added) was added continuously over the next 1.5 hours. The reaction temperature was maintained between 50-60° C. with an ice/water bath. The ice bath was removed during the last 10% of the addition. Once the last of the bromide was added, the reaction was allowed to stir for 1.5 hours, during which time the temperature dropped to 35° C. At this point, there was very little unconsumed magnesium remaining, however the reaction solution was heated to 60° C. for 30 minutes to ensure completion. The reaction was cooled to −10° C. and excess carbon dioxide was added into the reaction mixture through the condenser. The reaction became quite thick and the temperature rose to ˜30° C. At this point an additional IL of THF was added. The carbon dioxide was added until the reaction was complete and the temperature began to drop. A total volume of 350 mL of THF was removed under reduced pressure. The resulting thick slurry was quenched with a mixture of 4.4 L of ice cold water and 320 mL concentrated HCl. To the resulting thick white slurry an additional 4 L water was added. The resulting precipitate was filtered and washed with 1.5 L water, dried on the funnel overnight, and dried at 60° C. under high vacuum to provide 386.05 g of the desired product as a white powder. HPLC showed a peak at the expected retention time 15.67 min and a purity of 97.45% at 220 nm and 98.98% at 254 nm.

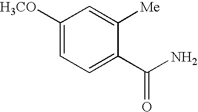

### Example 1B

### 4-methoxy-2-methylbenzamide

A mixture of Example 1A (386.05 g) in dichloromethane (3 L) was combined in a flask equipped with a mechanical stirrer, reflux condenser, and addition funnel to provide a very thick slurry. DMF (1 mL) was added as catalyst, followed by oxalyl chloride (330 g) dropwise over about 2 hours. The acidic effluent gases were scrubbed through a K₂CO₃ scrubber. The slurry slowly dissolved during the addition to provide a red solution. Dichloromethane (1.3 L) was distilled off at 30° C. with slight vacuum, and the resulting concentrated solution of acid chloride was polish filtered through a course sintered glass funnel to remove some insoluble matter. This filtered solution was concentrated to a crystalline residue and concentrated under high vacuum for 30 minutes to remove any excess oxalyl chloride. The crystalline residue was dissolved in THF (550 mL) and titrated into a large flask containing ice cold concentrated ammonium hydroxide (IL) over ˜15 minutes. The temperature quickly rose to ˜30° C. with the formation of a thick slurry of product. To this oily slurry of product, water (3 L) was added over ˜15 minutes to provide a thick white slurry of product. This product was filtered over course sintered glass and washed with water (1.5 L) and dried under nitrogen/vacuum for 36 hours. The desired product was isolated (367.8 g) as an off-white solid. HPLC showed a peak at the expected retention time of 11.85 min, with a purity of 95.15% at 220 nm, and 97.29% at 254 nm.

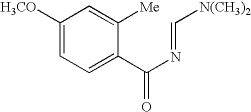

### Example 1C

### 4-methoxy-2-methylbenzoyl(N,N-dimethyl)formamidine

Example 1B (296.15 g, 1.794 moles) was dissolved in THF (1.5 L) in a flask equipped with a mechanical stirrer and a distillation head to give a thick slurry. DMF-DMA (263 mL, 1.1 eq) was added in one portion and slowly heated to gentle reflux. After 30 minutes at reflux, the reaction mixture became a homogeneous solution. The reaction was maintained at reflux for 1.5 hours, and checked by HPLC and TLC (10:1 CH₂Cl₂/CH₃OH). At atmospheric pressure, 1150 mL THF was distilled out and replaced with 1500 mL heptane (Note that if the solution is not quite saturated, remaining THF should be removed by distillation). The remaining solution was cooled slowly to room temperature overnight with stirring and seeded at 68° C. Rapid crystallization was observed. The resulting slurry was cooled to 0° C., filtered, and the solid washed with heptanes (500 mL) and dried under vacuum at room temperature for 48 hours. The desired product was isolated (384.6 g, 97.4%) as a light tan crystalline solid. HPLC-MS using a neutral buffer ammonium acetate buffer system showed only a single peak, with the expected mass.

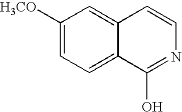

### Example 1 D

### 6-methoxyisoquinolin-1-ol

A slurry of Example 1C (16.33 g) in a small amount of THF (25 mL) was heated to 60° C. in a flask equipped with a stir bar, reflux condenser, and addition funnel. A solution of (IM potassium tert-butoxide in THF, 105 mL) was titrated in over a period of 30 minutes. The reaction turned a light yellow, and began to precipitate a solid product after about 10 minutes. The mixture became a thick suspension after 30 minutes. The reaction was cooled to ˜30° C. and neutralized to pH 7 with 9.5 mL conc. HCl. Water (about 25 mL) was added to dissolve all the salts, and still an easy phase split remained. The phases were split and the aqueous phase was back extracted with 25 mL ethyl acetate. The organic phases were combined and slowly concentrated at a temperature of about 60° C. to provide a crystalline residue. The desired product was isolated as a light orange solid (12.16 g, 93.6%). An analytical sample was prepared by recrystallization from ethyl acetate. LC retention time=13.533 min. ¹H NMR (DMSO-d₆) δ 3.86 (s, 3H); 6.46 (d, 1H); 7.02-7.13 (m, 3H), 8.08 (d, 1H); 11.03 (s, 1H).

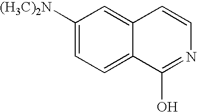

### Example 2

### Preparation of 6-(N,N-dimethylamino)isoquinolin-1-ol

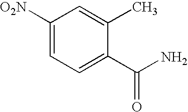

### Example 2A

### 4-nitro-2-methylbenzamide

A suspension of 2-methyl-4-nitrobenzoic acid (50.0 g, 276.02 mmol), in dichloromethane (200 mL) at room temperature was treated with oxalyl chloride (30.1 mL, 345 mmol) and a few drops of DMF. After 1 hour another 5 drops of DMF were added. After a total of three hours the mix was concentrated under vacuum.

A solution of the concentrate in THF (80 mL) was added to ice-cold aqueous concentrated NH₄OH (100 mL) at 0° C. with rapid stirring. When the addition was complete, water (200 mL) was added and the mixture was stirred at 0° C. for 30 minutes. The resulting solid was collected by filtration and dried under vacuum at 45° C. for several hours to provide 46.38 g (93.3%) of the desired product as a slightly yellow powder. LCMS shows m/z at 181 (M+H)⁺.

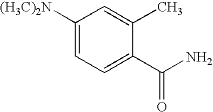

### Example 2B

### 4-(N,N-dimethylamino)-2-methylbenzamide

A mixture of Example 2A (10.0 g, 55.5 mmol) in 95% ethanol (150 mL) was heated with a heat gun to provide a solution. The mixture was cooled to room temperature by placing in an ice bath, flushed with nitrogen, and treated with 10% Pd/C (600 mg). The mixture was placed on a Parr hydrogenator and hydrogen was added very slowly. The mixture became quite warm over about 20 minutes and stopped consuming hydrogen after about 20 minutes. The mixture was shaken at 55 psi hydrogen for an additional 1.5 hours. LCMS showed complete hydrogenation.

Aqueous formaldehyde (18 mL, 4 equiv.) was added and the mixture was placed back on the Parr shaker and pressurized to 55 psi. After about 2 hours the mixture stopped consuming hydrogen. The mixture was allowed to sit under nitrogen overnight, then heated under nitrogen until all of the solids dissolved. The catalyst was removed by filtration and the filtrate was concentrated to a final volume of approximately 75 mL while heating to about 65° C. The mixture was heated until all solids dissolved, cooled to room temperature, placed in a freezer after 1 hour, and then filtered. The solid was rinsed with ice-cold ethanol to provide white crystals (6.057 g) that was shown to be the desired product that was about 97+% pure.

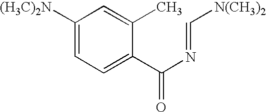

### Example 2C

### 4-(N,N-dimethylamino)-2-methylbenzoyl(N,N-dimethyl)formamidine

A mixture of Example 1B (6.0 g, 33.7 mmol) and DMF-DMA (5.37 mL, 40.4 mmol) in THF (30 mL) in a flask equipped with a short path distillation head was heated to boiling. After about 30 minutes the distillation head was replaced with a reflux condenser. The mixture was heated to reflux. After about 3.5 hours the mixture was concentrated under vacuum to provide a yellow solid that was dried under vacuum at about 65° C. for about 2 hours to provide 7.61 g of the desired product.

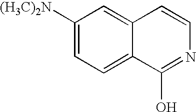

### Example 2D

### 6-(N,N-dimethylamino)isoquinolin-1-ol

A mixture of Example 2C (7.1 g, 30.4 mmol) in THF (15 mL) was treated with potassium tert-butoxide (1.0M in THF, 46 mL, 46 mmol) and heated to reflux for about 18 hours. The mixture was removed from heat and treated with 12N HCl (4 drops). Additional HCl was added until the pH reached 7. The mixture was concentrated under vacuum, dissolved in hot methanol (100 mL), treated with ethyl acetate (100 mL), and filtered. The filtrate was concentrated to provide 7.06 g of the desired product as a yellow solid. LC-MS (retention times: 0.553 min, 1.202 min (tautomers)), MS m/z 189 (M⁺+1) for both peaks.

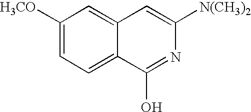

### Example 3

### 8-(N,N-dimethylamino)-6-methoxyisoquinolin-1-ol

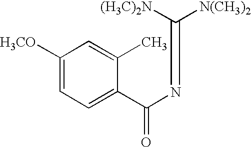

### Example 3A

### N″-(4-methoxy-2-methylbenzoyl)-N,N N′,N′-tetramethylguanidine

A solution of 4-methoxy-2-methylbenzoic acid (10.2 g, 61.4 mmol) in dichloromethane (200 mL) was treated slowly with oxalyl chloride. A drop of DMF was added and the reaction was stirred at room temperature for 12 hours. The solution was treated with 1,1,3,3-tetramethylguanidine (14.9 g, 128.9 mmol), stirred at room temperature for 4 hours, and washed with 5% aqueous citric acid (2×100 mL). The aqueous layer was extracted with dichloromethane (2×100 mL) and the combined organic phases were dried (MgSO₄), filtered, and concentrated to provide a minimal amount of the desired product. The aqueous layer was basified with ION NaOH to pH 12-13 and extracted with dichloromethane (4×200 mL). The combined organic phases were dried (MgSO₄), filtered, and concentrated. The resulting oil was combined with the earlier obtained product and treated with diethyl ether to form a white precipitate that was removed by filtration. The filtrate was concentrated to provide 11.32 g (70%) of the desired product as a yellow viscous oil. ¹H NMR (CD₃OD) δ 2.54 (s, 3H), 3.97 (s, 6H), 3.79 (s, 3H), 6.60 (d, J=9.2 Hz, 1H), 6.75 (s, 1H), 7.65 (d, J=8.9 Hz, 1H). LC-MS (retention time: 1.00 min), MS m/z 264 (M⁺1).

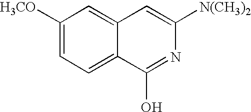

### Example 3B

### 8-(N,N-dimethylamino)-6-methoxyisoquinolin-1-ol

A solution of Example 3A (461 mg, 1.78 mmol) in THF (5 mL) at 0° C. was treated with LDA solution (1.8M in THF/hexanes, 1.2 mL, 2.136 mmol). The reaction was stirred at room temperature for about 5 hours and then treated with additional LDA (1 mL) and stirred at room temperature for 14 hours. The mixture was quenched with saturated ammonium chloride (10 mL) and extracted with ethyl acetate (3×25 mL). The combined organic layers were dried and concentrated. The concentrate was dissolved in minimal dichloromethane and diethyl ether was added. The resulting precipitate was collected by filtration to provide the desired product (312 mg, 80%) as a brown solid. ¹H NMR (CD₃OD) δ 2.94 (s, 6H), 3.86 (s, 3H), 5.71 (s, 1H), 6.75 (dd, J=8.9, 2.4 Hz, 1H), 6.83 (d, J=2.4 Hz, 1H), 7.97 (d, J=9.2 Hz, 1H). LC-MS (retention time: 1.26 min), MS m/z 219 (M⁺+1).

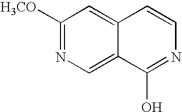

### Example 4

### Preparation of 6-methoxy-2,7-naphthyridin-1-ol

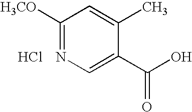

### Example 4A

### 6-methoxy-4-methylnicotinic acid hydrochloride

A solution of 5-bromo-2-methoxy-4-methylpyridine (23.6 g, 116.8 mmol) in THF (400 mL) at −78° C. was treated with n-butyllithium (2.5M in hexanes, 51.4 mL, 128.5 mmol) over 10 minutes via an addition funnel. The solution was stirred at −78° C. for 10 minutes then treated with excess CO₂ gas until the solution turned light yellow. The reaction was stirred for 1 hour and then quenched slowly with 1N aqueous HCl (60 mL) and water (40 mL). After warming to close to room temperature the layers were separated and the organic layer was extracted with water (2×100 mL) and 1N aqueous HCl (1×100 mL). The combined aqueous phases were concentrated to about ⅓ of the original volume and the resulting white precipitate was collected by vacuum filtration and washed with water and dried to provide 7.683 g of the desired product. The filtrate was concentrated to dryness and the light yellow solid was dried at 60° C. under vacuum to provide a mixture of product and LiCl. The combined solids were triturated with water, neutralized to pH 7 with NaOH, saturated with NaCl, and extracted with dichloromethane. The extracts were combined and concentrated to provide the desired product. ¹H NMR (DMSO-d₆) δ 2.50 (s, 3H), 3.88 (s, 3H), 6.73 (s, 1H), 8.64 (s, 1H), 12.75 (s, 1H). LC-MS (retention time: 0.382 min), MS m/z 166 (M⁺−1).

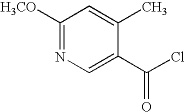

### Example 4B

### 6-methoxy-4-methylnicotinoyl chloride

A slurry of Example 4A (4.42 g, 26.44 mol) in dichloromethane (100 mL) was treated with oxalyl chloride (10.07 mg, 79.33 mmol) followed by DMF (102 μL). After 1 hour the reaction mixture was concentrated to provide the crude desired product.

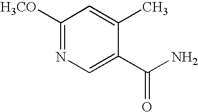

### Example 4C

### 6-methoxy-4-methylnicotinamide

A solution of Example 4B (2.1 g, 11.64 mmol) in THF (47 mL) at 0° C. was treated slowly with concentrated ammonium hydroxide. The ice bath was removed and the reaction was warmed to room temperature. After 30 minutes the resulting brown precipitate was collected by vacuum filtration and washed with water. The filtrate was diluted with ethyl acetate (50 mL), shaken, and the layers separated. The aqueous layer was extracted with ethyl acetate (4×50 mL) and the combined organic layers were dried (MgSO₄), filtered, and concentrated to provide the desired product. Both batches of solid were combined to provide 1.6 g (83%) of the desired product. LC-MS (retention time: 0.690 min), MS m/z 167 (M⁺1)

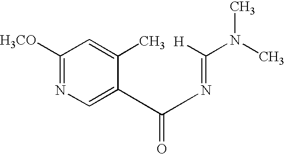

### Example 4D

### N—[(dimethylamino)methylidene]-6-methoxy-4-methylnicotinamide

A mixture of Example 4C (1.4 g, 8.42 mmol) and N,N-dimethylformamide dimethyl acetal (25 mL) in a medium pressure flask was heated to 115° C. for 1 hour and concentrated. The concentrate was dissolved in ethyl acetate (50 mL) and concentrated. The concentrate was dissolved in minimal dichloromethane and diethyl ether (100 mL) was added. The resulting precipitate was removed by filtration. The filtrate was concentrated to about ½ of its original volume and treated with pentane (150 mL). The mixture was slowly concentrated at room temperature to about ½ its original volume (˜100 mL) to provide a light brown solid precipitate. The solid was collected by vacuum filtration and washed with pentane. The filtrate was concentrated to provide additional product. The two solids were combined to provide 1.63 g (87%) of the desired product. ¹H NMR (CD₃OD) δ 2.61 (s, 3H), 3.16 (s, 3H), 3.17 (s, 3H), 3.95 (s, 3H), 6.5 (s, 1H), 8.55 (s, 1H), 9.08 (s, 1H). LC-MS (retention time: 0.965 min), MS m/z 222 (M⁺+1).

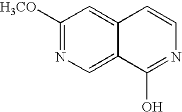

### Example 4E

### 6-methoxy-2,7-naphthyridin-1-ol

A solution of Example 4D (1.5 g, 6.78 mmol) in THF (30 mL) was treated with LDA solution slowly (1.8M in THF, 7.9 mL, 14.24 mmol). The resulting red slurry was stirred at room temperature for 1 hour and quenched slowly with 1N HCl (30 mL) then acidified to pH 5 with concentrated HCl. The mixture was diluted with ethyl acetate (50 mL) and shaken. The layers were separated and the aqueous layer was extracted with dichloromethane (3×50 mL). Some red precipitate formed and was collected by vacuum filtration and washed with dichloromethane. The filtrate was washed with brine and additional red solid precipitate formed and was collected by filtration. The organic phases were combined, dried (MgSO₄), filtered, and concentrated. Diethyl ether was added and a yellow solid formed. The precipitate was collected by vacuum filtration and dried in a vacuum oven at 50° C. overnight to provide the desired product. LC-MS (retention time: 1.55 min), MS m/z 177 (M⁺1).

What is claimed is:

1. A process for preparing a compound of formula (4) 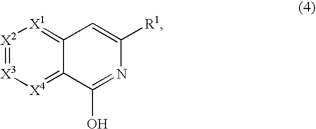
wherein
X¹, X², X³, and X⁴ are independently N or CR²; provided that if X¹ and X² are N then X³ is CR²; and provided that if X³ and X⁴ are N then X² is CR²;
R¹ is selected from hydrogen and —NR^{a}R^{b};
each R² is independently selected from alkenyl, alkoxy, alkoxyalkyl, alkyl, alkynyl, aryl, arylalkyl, halo, haloalkoxy, haloalkyl, heterocyclyl, heterocyclylalkyl, —NR^{a}R^{b}, and (NR^{a}K^{b})alkyl; and
R^{a} and K^{b} are independently selected from hydrogen, alkenyl, alkyl, alkynyl, aryl, and arylalkyl;
the process comprising:
(a) reacting a compound of formula (3) 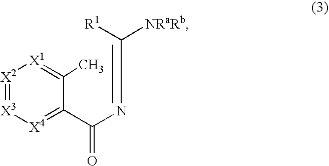
with a base to form a first reaction mixture;
(b) adjusting the pH of the first reaction mixture to about 7 to form a second reaction mixture; and
(c) optionally heating the second reaction mixture.

2. The process of claim 1 wherein step (a) is conducted in an organic solvent.

3. The process of claim 2 wherein the organic solvent is an ether.

4. The process of claim 3 wherein the organic solvent is tetrahydrofuran.

5. The process of claim 1 wherein the base is selected from potassium tert-butoxide, lithium tert-butoxide, sodium tert-butoxide, lithium hexamethyldisilazide, lithium diisopropylamide, and sodium hexamethyldisilazide.

6. The process of claim 5 wherein the base is potassium tert-butoxide.

7. The process of claim 5 wherein the base is lithium diisopropylamide.

8. The process of claim 1 wherein step (a) is conducted at a temperature of about 0° C. to about 70° C.

9. The process of claim 1 wherein step (a) is conducted for about 15 minutes to about 2 hours.

10. The process of claim 1 wherein the pH of the first reaction mixture is adjusted to about pH 7 with hydrochloric acid.

11. The process of claim 10 wherein the pH of the first reaction mixture is adjusted to about pH 7 with ammonium chloride.

12. The process of claim 1 wherein step (b) is conducted at a temperature of about 20° C. to about 40° C.

13. The process of claim 1 wherein step (c) is conducted at a temperature of about 50° C. to about 70° C.

14. The process of claim 1 wherein step (c) is conducted for about 15 minutes to about 2 hours.

15. A process for preparing a compound of formula (4a) 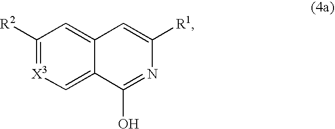
wherein
R¹ is hydrogen or —N(CH₃)₂;
R² is —OCH₃ or —N(CH₃)₂; and
X³ is CH or N;
the process comprising:
(a) reacting a compound of formula (3a) 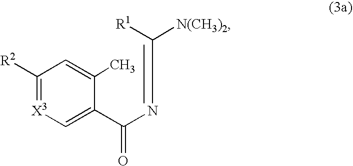
with a base to form a first reaction mixture;
(b) adjusting the pH of the first reaction mixture to about 7 to form a second reaction mixture; and
(c) optionally heating the second reaction mixture.

16. The process of claim 15 wherein
R¹ is hydrogen;
R² is —OCH₃; and
X³ is CH.
